(19) United States
(12) Patent Application Publication
(10) Pub. No.: US 2023/0123967 A1
(43) Pub. Date: Apr. 20, 2023

(54) DEVICE FOR THE REPETITIVE SUPPLY AND DRAINING OF SUBSTANCES FOR MEDICAL THERAPY, AND SYSTEM AND METHOD FOR PRODUCING FECAL CONTINENCE BY INTERMITTENT COLON LAVAGE BY MEANS OF A PERMANENTLY PLACED TRANS-ANAL OCCLUDING OR ACCESS CATHETER

(71) Applicant: Advanced Medical Balloons GmbH, Waghausel (DE)

(72) Inventor: Fred Göbel, Speyer (DE)

(21) Appl. No.: 17/993,312

(22) Filed: Nov. 23, 2022

Related U.S. Application Data
(63) Continuation-in-part of application No. 16/464,133, filed on May 24, 2019, now abandoned.

(30) Foreign Application Priority Data
Nov. 25, 2016 (DE) 10 2016 014 053.4

(51) Int. Cl. A61M 16/04; A61M 3/02

(52) U.S. Cl. CPC A61M 16/0463; A61M 3/0295; A61M 2210/1064

(57) ABSTRACT

A device for carrying out a lavage-like, repetitive supply and draining of a flushing, cleaning or other therapeutically active substance in the context of a return-flow enema, the device comprising: a catheter unit (SK) for receiving and draining feces, which can be permanently placed atraumatically and anorectally; a flushing and collecting bag unit (5, SB) with a combined flushing and collecting function; and a hose unit (4) connecting the catheter unit (SK) to the flushing and collecting bag unit (5, SB), comprising a single-lumen or multi-lumen, film-like, flatly collapsible, hose, and enabling a bidirectional flow communication between the catheter unit (SK) and the flushing and collecting bag unit (5; SB); characterized by at least one throughflow element with a flow-direction-dependent throttle function, providing a large lumen for draining of the patient's intestinal contents into the flushing and collecting bag unit (5; SB), so that shaped stool fractions can be drained, and which facilitates a supply of liquid from the flushing and collecting bag unit (5; SB) towards the patient, but wherein the supply of liquid towards the patient is throttled, so as to be reduced in flow amount or flow speed in relation to the draining of the patient's intestinal contents and thereby reduces the supply of liquid to the patient to a non-irritating amount that is compatible with the patient avoiding painful sensations and/or vegetative responses by an immediate dilation of the patient's colon and/or a triggering of a premature or only partially coordinated defecation reflex.

### REFERENCE TO PENDING PRIOR PATENT APPLICATIONS

This patent application is a continuation-in-part of pending prior U.S. patent application Ser. No. 16/464,133, filed 24 May 2019 by Advanced Medical Balloons GmbH and Fred Göbel for DEVICE FOR THE REPETITIVE SUPPLY AND DRAINING OF SUBSTANCES FOR MEDICAL THERAPY, AND SYSTEM AND METHOD FOR PRODUCING FAECAL CONTINENCE BY INTERMITTENT COLON LAVAGE BY MEANS OF A PERMANENTLY PLACED TRANS-ANAL OCCLUDING OR ACCESS CATHETER (Attorney's Docket No. KUCH-97), which patent application, in turn is a 371 national stage entry of prior International (PCT) Patent Application No. PCT/IB2017/001458, filed 27 Nov. 2017 by Advanced Medical Balloons GmbH for DEVICE FOR THE REPETITIVE SUPPLY AND DRAINING OF SUBSTANCES FOR MEDICAL THERAPY, AND SYSTEM AND METHOD FOR PRODUCING FECAL CONTINENCE BY INTERMITTENT COLON LAVAGE BY MEANS OF A PERMANENTLY PLACED TRANS-ANAL OCCLUDING OR ACCESS CATHETER, which patent application claims benefit of prior German Patent Application No. DE 10 2016 014 053.4, filed 25 Nov. 2016.

The three (3) above-identified patent applications are hereby incorporated herein by reference.

### FIELD OF THE INVENTION

The invention relates to a device, a system and a method for carrying out a lavage-like, repetitive supply and draining of a flushing, cleaning or other therapeutically active substance, in particular in the context of an evacuation by irrigation of a patient's colon.

Especially, a device for carrying out a lavage-like, repetitive supply and draining of a flushing, cleaning or other therapeutically active substance in the context of a return-flow enema, comprises:
a catheter unit for receiving and draining feces, which can be permanently placed atraumatically and anorectally;
a flushing and collecting bag unit with a combined flushing and collecting function; and
a hose unit connecting the catheter unit to the flushing and collecting bag unit, comprising a single-lumen or multi-lumen, film-like, flatly collapsible, hose.

The device has a bag unit or bag/hose unit which has a combined flushing and collecting function and also at least one flow-directing function which prevents flushing liquid which has already been drained into the bag from flowing back towards the patient in a direct manner and also allows shaped fractions to be deposited out of the flushing liquid in the bag.

The invention also relates to the combination of the bag unit or bag/hose unit with a catheter unit which can be placed in position in an atraumatic manner, is positioned trans-anally, seals off the anal canal, preferably by means of a dumbbell-shaped balloon component, and can be left in the patient's rectum, preferably after lavage is complete.

The permanently trans-anally placed catheter unit can be used for intermittent evacuation or lavage to keep the descending colon and rectum clear of feces and thus bring about a state of quasi-continence.

The method refers to the use of such a device for conducting a return-flow enema.

### BACKGROUND OF THE INVENTION

The document US 2011/0 160 657 A1 of Goebel discloses a device for sealing a natural or artificial opening of the colon or rectum of a patient and for a continuous or intermittent irrigation into an extracorporeal collection bag, the device comprising a catheter unit for placement inside of the rectum. On the other hand, the present invention refers to a device and a method for conducting a reverse-flow enema, for which procedure a flushing and collecting bag unit is needed instead of a collecting bag only. Within such flushing and collecting bag, a liquid for flushing the colon of a patient is provided, which flows into the colon, if the bag is raised above the level of the colon, and if subsequently the bag is lowered below the level of the colon, the liquid together with the intestinal contents of the patient is drained into the flushing and collecting bag. This is not possible with the arrangement of Goebel, either because no liquid is provided in the collecting bag which could be flushed into the patient's colon, and furthermore because a rearward flow of liquid is prevented in order to keep drained intestinal contents in the bag. Furthermore, even the drainage of formed stool portions requires a manual action with a press-out device in form of a barrette-like squeeze platelet with a keyhole-shaped opening comprising a circular portion with a narrow radial extension on one side. A hose connecting catheter unit with the collecting bag extends through this keyhole-shaped opening, and if the hose traverses the circular portion of the opening in the press-out device, the drainage lumen is completely open, whereas if the hose traverses the narrow radial extension of the opening, the hose is pressed together into a flat ribbon with no residual lumen. In this condition, the lumen inside of the hose is completely constricted to zero, and in order to drain the contents inside of the hose, the barrette-like squeeze platelet has to be moved by a manual action of the user along the hose. Onn the other hand, a continuous flow is not possible in such condition, and a manual action is rather complicated, making it impossible for a bedridden user to perform such a drainage for himself.

US 2010/0 280 491 A1 of Tanghoej refers to an irrigation system, the system comprising an insertion member for placement inside of a lumen of a patient, for example into the rectum of the patient, and an extracorporeal reservoir with an upper chamber and a lower chamber, which are connected in series and communicate with the insertion member via a hose or tube. At the upper end of the upper chamber, there is an inlet for pouring liquid into the reservoir. At this inlet, as well as between the two chambers and at the outlet of the lower chamber, there are provided one-way valves, which are only open in a direction from the reservoir to the patient, but are closed in the opposite direction. Therefore, a return-flow enema cannot is not possible with such an arrangement, as a draining of the liquid and of the patient's intestinal contents from the patient towards the reservoir is prevented by the one-way valves which are closed for this flow-direction.

Furthermore, this embodiment needs a manual pumping action to move the liquid from the lower chamber to the patient.

U.S. Pat. No. 4,801,292 B1 of Watson shows a medical pump for debris collection, without a catheter unit, a collecting bag and a hose connecting both. Instead of a catheter unit, this pump has a nozzle only. Due to the missing catheter unit inside of a patient and due to a lack of a hose connecting such catheter unit with the pump, it is not possible to lift and lower such pump, as it is the usual practice at a return-flow enema, because in such case, the contact with the rectum of a patient would get lost. Furthermore, as the pump has a rigid barrel, in contrast to a bag with a flexible sleeve, it cannot be used for a return-flow enema, where the volume inside of the bag varies if the liquid flows to the patient and if it returns—together with the patient's intestinal contents—into the bag. Instead, no liquid would flow out of such pump through its narrow nozzle, even if it would be raised to any level. The only way to pump liquid out of the nozzle is to squeeze a pump balloon at the rearward end of the pump.

Therefore, in the entire state of the art, there exists either a collection bag or a flushing or irrigation reservoir, but no cimbination, and therefore, two different systems have to be used in case of a return-flow enema—one for flushing the colon of a patient with a liquid and the other for collecting the intestinal contents of the patient. Furthermore, complex manual operation is needed, for flushing a liquid into the colon of a patient as well as for the subsequent drainage of the patient's intestinal contents.

In contrast, the present invention refers to a device and a method for an irrigation of a patient's colon or rectum by means of combined supply and draining catheter systems.

Furthermore, previously known designs of flushing systems for colorectal irrigation are unsatisfactory in many respects. In particular, in the case of conventional irrigation systems, the catheter part placed trans-anally in the rectum must be removed immediately following the colorectal irrigation. Due to the normally rigid design of the catheter shaft that supports comfortable trans-anal insertion, it is possible for a traumatic perforation of the wall of the intestines to occur in the event of dislocations of the catheter directed towards the intestines. The unguaranteed option of long-term positioning of the catheter in the rectum which precludes trauma is problematic in clinical use in this respect, since even though the intestinal contents are mobilized by the irrigation, they are frequently only evacuated in many subsequent fractions following the irrigation. The subsequent bowel movements persisting over many hours normally go hand in hand with substantial soiling of the patient.

So-called return-flow enemas are normally carried out to clean or evacuate the intestines or to mobilize solid stool from a patient's rectum or colon. With this method, irrigation liquid is filled into a container, which is connected to an irrigation catheter normally secured with a retaining balloon and which is inserted trans-anally and extends into the rectum and is placed there via a hose system. The container is lifted above the patient's level to supply the medium and lowered below the patient's level to drain the mobilized intestinal contents. The method is repeated until the stool to be washed out of the intestines has dissolved or is suspended in the irrigation medium enough that it can be drained into the receiving container as much as possible during the concluding evacuation of the intestinal contents. Several successive lifting and lowering cycles are required for an efficient suspension of stool, wherein the method is normally protracted. In addition, when the cycle is repeated multiple times, irrigation liquid increasingly gets into higher sections of the colon, which leads to a further delay in eliminating the mobilized intestinal contents.

As a result, a user-friendly system for conducting a return-flow enema that minimizes the number of required flushing and draining cycles as much as possible would be desirable. The system should be suitable as a whole for a rapid and efficient washing out of intestinal contents, in particular also of formed portions of stool. Furthermore, it should also be possible for the irrigation system to remain in the patient's rectum after the end of the irrigation process over several hours to possibly even days in order to be able to receive intestinal contents mobilized by the irrigation that are eliminated in a delayed manner and/or to guide them into a closed collection system. Therefore, a usage-optimized device or system without a complex manual handling would be advantageous.

### SUMMARY OF THE INVENTION

The solution to this problem succeeds in the context of a generic device, comprising a catheter unit for receiving and draining feces, which can be permanently placed atraumatically and anorectally, and passes through and seals the anal canal preferably over the entire length thereof, a flushing and collecting bag unit with a combined flushing and collecting function, as well as a hose unit connecting the catheter unit to the flushing and collecting bag unit, comprising a single-lumen or multi-lumen, film-like, flatly collapsible hose, and enabling a bidirectional flow communication between the catheter unit and the flushing and collecting bag unit by at least one through-flow element with a flow-direction-dependent throttling function, providing a large lumen for the draining of the patient's intestinal contents into the flushing and collecting bag unit, so that shaped stool fractions can be drained, and which facilitates a supply of liquid from the flushing and collecting bag unit towards the patient, but wherein the supply of liquid towards the patient is throttled so as to be reduced in flow amount or flow speed in relation to the draining of the patient's intestinal contents and thereby reduces the supply of liquid to the patient to a non-irritating amount that is compatible with the patient.

Thereby, the inward flow of liquid to the patient is throttled to an atraumatic, non-irritating amount that is compatible with the organ and reduces or rules out the unchecked, high-volume return flow of ligation liquid already drained into the bag to the patient through a draining hose lumen. Such throttling of the inward flow of liquid to the patient has several advantages. First, the patient is reprieved from painful sensations; second, vegetative responses, for example cardiac arrythmias, a raise of the blood pressure, or sweating attacks, which could be cuased by an immediate dilation of the patient's colon, are avoided, too; third, a triggering of a premature or only partially coordinated defecation reflex is prevented.

As a part of the application, the term “placeable in an atraumatic manner” canishould be interpreted to the effect that it is free of a rigid, shaft-like supporting body.

Furthermore, the term “placeable in an anorectal manner” can/should signify that the body in question is placed in the rectum through the anus.

The term “through-flow element” describes an element which is permanently open for a through-flow in both directions, so that—dependent from the actual pressure direction—a flow of liquid can occur at any time in any one of the two possible flow directions. Such “through-flow element” according to the present invention does not predetermine the direction of flow, but has only a limited influence on the amount or speed of flow—limited insofar as it is never able to prevent a flow of liquid—neither in the one direction nor in the other direction. Furthermore, the “through-flow element” according to the present invention operates automatically—that means that a manual operation is neither necessary nor possible. Therefore, in this sense, the structure of such “through-flow element” according to the present invention is comparable to a hose, tube, pipe, duct or conduit which allows a flow in both directions at any time, dependent on the actual pressure direction. For this reason, a “through-flow element” according to the present invention is never entirely closed, neither in the one direction nor in the other direction, and especially, it is not closeable by a manual operation. In order to reduce the complexity of the handling, the function of such through flow element in its entirety shall be automatic, that means without any manual step or activity. Furthermore, the “through-flow element” shall be inserted into the flow connection between the catheter unit on the one hand and the flushing and collecting bag unit at the other hand at a fixed position, without any movement. Like the hose unit, the “through-flow element” does not impair a bidirectional flow communication between the catheter unit and the the flushing and collecting bag unit.

“Flow-directing” shall be understood as a function which allows different flow channels, or flow amounts, depending on the flow direction.

“Throttling” shall be understood as a limited and gradual reduction in the flow amount and/or flow speed, to a reduced flow amount or flow speed greater as zero. An entire disruption of the flow in any direction like it is done by a check valve is no throttling in the sense of the present patent application. Furthermore, the term “throttling” shall be undestood as a permanent function and not as a switching element like a valve.

This structure according to the invention makes it possible to use the same bag to receive the irrigation liquid and to receive the washed-out intestinal contents, because a return flow of already washed-out stool fractions to the patient is completely or largely ruled out.

For the efficient drainage of shaped stool actions, all segments of the catheter system are preferably equipped with a diameter large enough for the passage of the stool. As a part of the invention, functional components are described which separate the shaped stool fractions from the repetitively supplied irrigation medium in the system so that only aqueously suspended stool flows back to the patient. The efficient diversion and separation of shaped stool portions reduces the number of irrigation cycles substantially hi that the stool fragments do not have to be brought to the patient's intestines in an aqueous suspension in order to be drained, as required with conventional systems that normally have small-lumen catheter shafts.

A complete or partial separation of supplied irrigation medium and drained intestinal contents can take place as a result of corresponding functional elements both in the head-part segment, in the trans-anally placed segment, in the supplying or draining hose segment between the head part and the container, or can even be integrated into the container itself.

Colorectally irrigating catheter systems that are designed to be large lumen and are placed trans-anally are known, however, they are problematic in their application especially in the region of the trans-anal catheter segment. The typically relatively rigidly designed shaft elements thereof dilate the anal sphincter muscle and also go hand in hand with a risk of perforating the exposed sections of the intestines.

An influx of irrigation medium to the intestines that is too rapid and cannot be modulated is also problematic in the case of large-lumen irrigation and enema systems. If too great a volume flows too rapidly into the intestines, this can trigger painful sensations and even spasmodic contractions. As a result, a function restricting the flow towards the patient would be desirable, which, in the simplest case, can be facilitated by a throttling dimensioning in the supplying components.

A further problem with conventional colorectal irrigations is the intake of larger flushing volumes.

Many patients cannot safely accommodate large intake quantities in their intestines, e.g., due to a poor general state of health or because of a weak sphincter muscle. Even though the patient can initially prevent a leakage of the intestinal contents by contracting the sphincter muscle, the sphincter generally becomes fatigued after several minutes. Then it is possible for the intestinal contents to run out past the trans-anally placed shaft of the irrigation catheter.

In the preferred embodiment of the present invention, the catheter has a head unit, which is designed to stay permanently and atraumatically in the rectum. Commercially available stool drainage or fecal management systems are a possibility as a basic design type for continuous stool drainage in this case. These systems have a simple retention balloon with a toroidal design which rests anchored on the base of the rectum. In its extension, the balloon is connected to a simple trans-anally feces-draining hose, which is radially folded over or inside out in the anal canal.

Furthermore, head units with a special trans-anal balloon seal can be used in the context of the invention such as those described for example in EP 1 784 140 B1, WO 2013/026 564 A1 or DE 10 2008 055 674 A1. In addition to a dumbbell-shaped balloon for combined rectal retention and trans-anal sealing, said head units also have a trans-anally positioned, axially and radially compressible shaft component supporting the balloon, which shaft component receives the stool rectally and guides it through the anus. The described design has proven to be especially atraumatic and allows dwell times of several hours to days. In addition, the head units can be adapted to the respective sphincter opening and thereby ensure a dynamic acting, very efficient seal of the anal canal.

According to the invention, an extracorporeal supply and draining hose connects to the head unit, and said hose is terminally connected to a flushing and collecting bag. In its preferred embodiment, the draining hose is thin hi afilm-like manner so that, even in a filled state, a large drainage lumen can be achieved, but in an unfilled state, the hose collapses into a flat, strip-like structure and thereby optimally causes little irritation to the patient and prevents pressure spots in the event that the patient lies on the hose.

A possible way to realize a through-flow element with a flow-directing or a flow-direction-dependent throttling function would be to provide two different flow channels or flow lumens, one for each possible flow direction, and to equip at least one of both with a reduced diameter or cross-section in order to throttle the flow through this passage. In order to direct the flow of liquid to the regarding channel—dependent from the pressure direction which drives the flow of liquid—several possibilities exist.

First, at least a channel or lumen with an unthrottled or not reduced cross-section could be provided with a closeable element with a function like a one-way valve, but which—contrary to a common one-way valve—is able to allow the passage of larger formed stool portions.

Another possibility would be to place the inlet and outlet ends of both channels or lumens at the same end of the connecting hose between the catheter unit and the flushing and collecting bag unit spaced from each other, especially in such way that an outlet-only termination is not in the range of the regarding liquid.

The hose segment connecting the head unit to the flushing and collecting bag unit can have one or a plurality of lumens. Said lumens can be arranged, for example, concentrically to each other or even be disposed in parallel. Both structures are preferably made of a collapsible tubular film structure. The double-lumen arrangement makes it possible to completely separate the supplied liquid from the drained intestinal contents. In this special case, both lumens terminate at the distal end of the device at approximately the same height. If flow-directing components are used in the respective supply and draining hose portions, the supply liquid can be virtually completely protected from being contaminated by already drained intestinal contents or by surfaces contaminated with intestinal contents.

The container that stores the intake liquid or receives the intestinal contents eliminated by the patient constitutes an essential component of the flushing system presented in the invention. The invention proposes both single-chamber as well as double-chamber systems in this case.

In the embodiment with one chamber, a feces-separating system is preferably provided, which retains shaped stool fractions and only allows the inflow of suspended stool particles to the intestines. The container functions so to speak as a stool separator. In the two-chamber design, one compartment of the container contains the irrigation liquid, while the other compartment, which is completely separated from the first one, receives the drained intestinal contents.

Flow-directing valve mechanisms are provided in both the single-chamber as well as the double-chamber container system to direct the respective flows double. A flow-directing function can optionally be Integrated into the connecting hose system between the catheter element and the container. Installation in the trans-anally placed head unit is also conceivable. In the case of the components to be used, the invention starts with especially lightweight, atraumatically collapsible, preferably film-like, components having a flow-directing function.

The invention describes a device and a method for carrying out a lavage-like, repetitive supply and draining of a flushing, cleaning or other therapeutically active substance, in particular in the context of an evacuation by irrigation of a patient's colon, wherein the device has a bag or bag/hose unit with a combined flushing and collecting function, which has at least a flow-directing function, which rules out the direct return flow of irrigation liquid that has already been drained into the bag to the patient, and also allows the deposit of shaped fractions from the already drained flushing liquid in the bag.

The combination of the bag or bag/hose unit according to the invention with an atraumatic, trans-anally placed catheter unit, which seals the anal canal preferably with a dumbbell-shaped balloon component, makes large-volume inflows possible which enable a virtually complete evacuation or lavage of the colon without causing irrigation liquid to leak out of anus during the inflow. The atraumatic placeability of the catheter unit furthermore allows the catheter to be left in the patient's rectum after lavage is complete, and not to be removed until the colon is completely evacuated, whereby soiling of the patient from subsequent stool evacuations can be prevented. In the case of the permanent trans-anal placement of the catheter unit, it is possible for it to be used for an intermittent evacuation or lavage, which keeps the descending colon or the rectum clear of feces, and thus facilitates the status of a quasi-continence. In the interval between irrigations, the catheter unit placed in the anus ensures a continuous sealing of the anus. It is then terminated with an especially lightweight, film-like, degassing and/or secretion-collecting element. The connection of the irrigating bag/hose unit as well as the terminating, degassing units is accomplished with an atraumatic, flatly collapsible connector unit, which prevents the development of pressure spots,

A method according to the present invention for carrying out a lavage-like, repetitive supply and draining of a flushing, cleaning or other therapeutically active substance in the context of a return-flow enema, comprises the following steps:
a) providing a device comprising:
   a catheter unit for receiving and draining feces, which can be permanently placed atraumatically and anorectally;
   a flushing and collecting bag unit with a combined flushing and collecting function;
   a hose unit connectable between the catheter unit and the flushing and collecting bag unit, comprising a single-lumen or multi-lumen, film-like, flatly collapsible, hose and enabling a bidirectional flow communication between the catheter unit and the flushing and collecting bag unit; and
   at least one throughflow element with a flow-direction-dependent throttle function, wherein the throughflow element is arranged in one of the catheter unit, the flushing and collecting bag unit or the hose unit or between two of them, and comprises a large lumen for draining of the patient's intestinal contents into the flushing and collecting bag unit, so that shaped stool fractions can be drained, and facilitates a supply of liquid from the flushing and collecting bag unit towards the patient, but wherein the supply of liquid towards the patient is throttled, so as to be reduced in flow amount or flow speed in relation to the draining of the patient's intestinal contents and thereby reduces the supply of liquid to the patient to a non-irritating amount that is compatible with the patient avoiding painful sensations and/or vegetative responses by an immediate dilation of the patient's colon and/or a triggering of a premature or only partially coordinated defecation reflex;
b) insertion of the the catheter unit into the natural or artificial opening into the colon of the patient;
c) connecting the flushing and collecting bag unit to the catheter unit (SK) via the hose unit and the throughflow element;
d) supplying liquid from the flushing and collecting bag unit towards the patient by raising the flushing and collecting bag unit to a level above the intestine of the patient, wherein the supply of liquid towards the patient is throttled by the throughflow element, so as to be reduced in flow amount or flow speed in relation to the draining of the patient's intestinal contents and thereby reduces the supply of liquid to the patient to a non-irritating amount that is compatible with the patient;
e) draining liquid and the patient's intestinal contents from the patient to the flushing and collecting bag unit by lowering the flushing and collecting bag unit to a level beneath the intestine of the patient, wherein the throughflow element provides a large lumen for draining shaped stool fractions together with the other intestinal contents of the patient;
f) repeating the above steps d) and e) until the descending colon of the patient is evacuated of feces.

By this method, the descending colon and even portions of the traverse colon can be evacuated from feces, in order to bring the patient into a condition of quasi-continence for several hours.

An additional, preferred application of the present invention is, for example, the therapy of a so-called hepatic encephalopathy as a specific, therapeutic method. The clinical symptoms develop within the context of a heavy functional disorder of the liver, whereby the liver filfills its metabolic, detoxifying functions only partially. In case of a degradation of different amino acids in the colon of a patient, for example ammonia occurs, which can achieve a critical level in the blood in case of a failure of the liver. Such a critical level of ammonia in the blood can lead to a swelling of predetermined types of cells in the brain, and this in turn may lead to a typical cascade of neurological symptoms.

An established form of therapy of the hepatic encephalopathy is the rectal infusion or instillation of a solution of lactulose and/or lactitol. Lactulose has an influence on the intetinal flora in the sense of a quantitative preponderance of lactic acid producing intestinal bacteria, whereby proteolytic, ammonia producing intestinal bacteria are repressed, and furthermore, the generation of ammonia is inhibited by an enzymatic blocking. By the resulting low pH value, Ammonia is protonated to ammonium which is discharged as a salt.

In many cases, the rectal infusion of typically lactulose is preceded by a purging step, wherein ammonia producing intestinal flora is drained from the colon in a natural way. Subsequent to the purging step, a liquid containing lactulose is introduced into the rectum, whereby the ammonia producing metabolism of the proteolytic flora is inhibited.

While at rectal enema systems of conventional design, only a limited evacuation of intestinal contents can be achieved, and neither an infusion of higher volumes nor a temporarily determined instillation of lactulose in the colon of a patient can be ensured, this will be facilitated by the device and method according to the present invention.

The method starts with evacuation of the colon by an enema with a high volume of liquid, especially with high rates of fluid flow which are ensured by the design of the device according to the invention. Thereby, beyond the sigma, even the descending colon and the transverse colon can be reached. Formed and partially formed stool portions from these higher sections of the colon can be mobilised by a flushing introduction of liquid, and can be washed out from the colon by a repetitive cycle of alternating raising and lowering. Ideally, even this purging lavage is conducted with a therapeutically concentrated lactulose preparation.

Subsequent to the evacuating lavage of the colon, a freshly prepared lactulose solution is infused into the colon, wherein the introduced volume can substantially exceed conventional volumina of 100 to 300 ml, and can thereby reach a higher amount of the flora remaining in the colon. Within the scope of the method according to the present invention, the infused volume can be increased up to 1500 ml. Furthermore, the user can determine the time of exposure of the solution in the colon, by temporarily closing the feeding hose unit accordingly.

Preferably, the intracorporeal head unit of the device according to the present invention comprises a reliable trans-anally sealing type of construction, which adapts to the relevant motility of the sphincter and thereby ensures a continuous sealing effect or, respectively, prevents a contamination of the environment of the patient by intestinal contents. Especially, this is achieved by use of one of the head units disclosed in the drawing.

The metabolic active efficiency of a therapeutic enema with lactulose can be substantially improved by the possibility of a non-leaking, contamination-free mobilising and removal of intestinal contents of higher poritions of the colon, and by the subsequent non-leaking, temporarily determinable instillation of high volumes of lactulose solution.

### BRIEF DESCRIPTION OF THE DRAWINGS

Other features, details, advantages, effects and functional aspects of the invention are yielded from the following description of several embodiments of the invention as well as on the basis of the attached drawings, wherein:

FIG. 1 shows a device according to the invention with a single-chamber, combined irrigation and collecting container with flow-direction-separated supply and draining, as well as with a retention unit for receiving the stool and anchoring the device in the rectum;

FIG. 2a discloses a double-lumen hose element made of thin-walled, easily collapsible tubular film, having a parallel progression of the individual lumens;

FIG. 2b shows a double-lumen hose element made of thin-walled, easily collapsible tubular film, with a concentric progression of the individual lumens;

FIG. 2c shows another embodiment of a hose element, partially truncated, for connecting a head unit of the catheter system to a combined flushing and collecting bag, wherein the hose element comprises an element with a flow-direction-dependent throttle function;

FIG. 3 discloses a preferred single-charr ber embodiment of the device according to the invention for the repetitive supply and draining of a substance, with an integrated deposit function of shaped elements already drained into the collecting container, in conjunction with a preferred trans-anally sealing catheter unit;

FIG. 4 shows a completely separated, double-chamber flushingicollecting bag system with integrated flow-directing functions in the outlet region of the bag;

FIG. 5a discloses an element of a set-based embodiment of a device according to the invention, which is designed in particular for the continuous fecal management of permanently immobile, incontinent patients with normal stool characteristics, wherein a special method for intermittent trans-anal colon irrigation to establish a quasi-continence of patients will be described in the context of the figure, wherein the individual components of this set are depicted in FIGS. 5a to 5e. In this case, FIG. 5a shows a flushing and collecting bag with an integrated feces-separating and flow-directing function and terminal connector;

FIG. 5b shows a lightweight, proximally terminating collecting and degassing bag;

FIG. 5c discloses a terminating element filled with a gauze-like material and having a degassing function;

FIG. 5d shows a special embodiment of a trans-anally placed head unit having a trans-anally tapered, feces-separating shaft element formed in an undulated manner;

FIG. 5e discloses an axially pluggable, flatly collapsible connector with an axially acting lock function;

FIG. 6 shows a simple embodiment of the head unit of a device according to the invention;

FIG. 7 discloses a further embodiment of the invention, which has a separate, trans-anally running, thin-walled tube or hose piece, which continuously connects the funnel to the preanal connector;

FIG. 8 shows a combination of the anchor balloon with a trans-anal segment according to FIG. 6 emanating from the anchor balloon sleeve, with the stabilizing tube piece depicted in FIG. 7;

FIG. 9 discloses a modified embodiment of the invention, whose trans-anal segment has two concentric hose layers;

FIG. 10 shows an embodiment of the invention that is modified in turn, which combines the previous embodiment according to FIG. 9 with the elastically deformable, self-straightening and aligning, tubular mesh depicted in FIG. 8;

FIG. 11 discloses a perspective view of a further embodiment of the invention; and

FIG. 12 shows a longitudinal section through FIG. 11 along Line XII-XII.

### DETAILED DESCRIPTION OF THE PREFERRED EMBODIMENTS

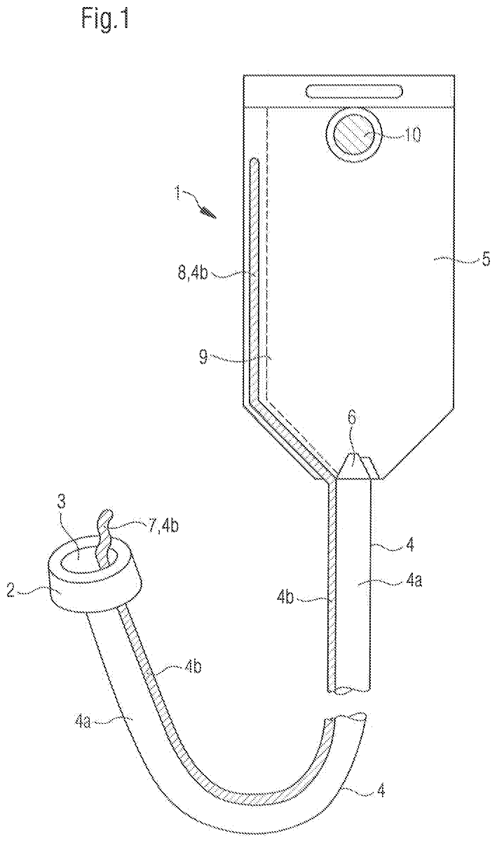

In the context of the invention, FIG. 1 shows an exemplary, single-chamber flushing catheter system 1 for the flow-direction-separated, feces-separating supply of liquid in the patient's rectum or colon as well as for draining mobilized intestinal contents. Located at the forward end of the catheter is a preferably flat, toroidal retention balloon 2, which forms a large-lumen central opening 3 having a diameter of preferably 15 to 30 mm for the intra-rectal reception of liquid or fiowable stool, Attached to the anchor balloon towards the proximal is a thin-walled, preferably flatly collapsible tubular film element 4 that integrates two parallel running lumens and connects the part of the device inside the patient to the flushing and collecting bag 5 situated outside of the patient. The lumen 4b supplying the irrigation liquid has a relatively smaller diameter, which reduces or throttles the flow of supplied irrigation liquid in order to prevent irritations from inflow that is too rapid and/or an associated abrupt dilation of the rectum. On the other hand, the lumen 4a draining the intestinal contents has a diameter whose dimensions are large enough to allow an outflow of shaped stool fragments into the container or bag 5 that is as unimpaired as possible. The special functional principle of the device is based on the integration of flow-directing and feces-separating elements or functions, which overall prevents shaped stool fractions that are already in the bag from being flushed back into the intestines during a subsequent inflow phase. To this end, a valve-like unit 6 is attached, for example as a flap valve or lip valve, in the leg 4a draining from the patient, preferably in the transition area of the draining leg to the bag. A corresponding flow-direction-separating valve function in the supplying leg 4b is preferably established in the region of the patient-side outlet of the leg by a functional element 7, which is constituted for example as a thin-walled, easily collapsible hose connecting piece, which opens with force acting from the inside and closes with force acting from the outside. Alternatively, the elements 6 and 7 can also be installed in other segments of the hose systems or even in the container itself.

To separate or deposit already drained, flaky or shaped stool fractions from the bag contents, the device is provided with a depositing inlet element 8, which projects into the interior of the bag preferably up to the upper rim of the bag and ensures that already drained shaped stool fractions are prevented from reentering the intestines during supply phases. The element 8 can be designed for example as a tubular-mesh-like structure or even as a sieve-like tube with multiple perforations. To affix and protect the inlet element in the bag, the element 8 can also be enclosed by a separating film 9 inside the bag, which is likewise perforated, sieve-like or mesh-like, the perforations of which are in turn preferably larger than the perforations of the element, whereby especially large, shaped stool fragments are prevented from infiltrating into the compartment accommodating the element. The occlusion of the inlet element via stool from the repetitive lifting and lowering inflow is prevented in the depicted design by the combination of an inlet element extending an especially long way inside the bag up to the upper rim of the bag as well as by an additional filtering function upstream from the inlet of the supplied liquid. The single-chamber bag is filled through a sealable opening 10.

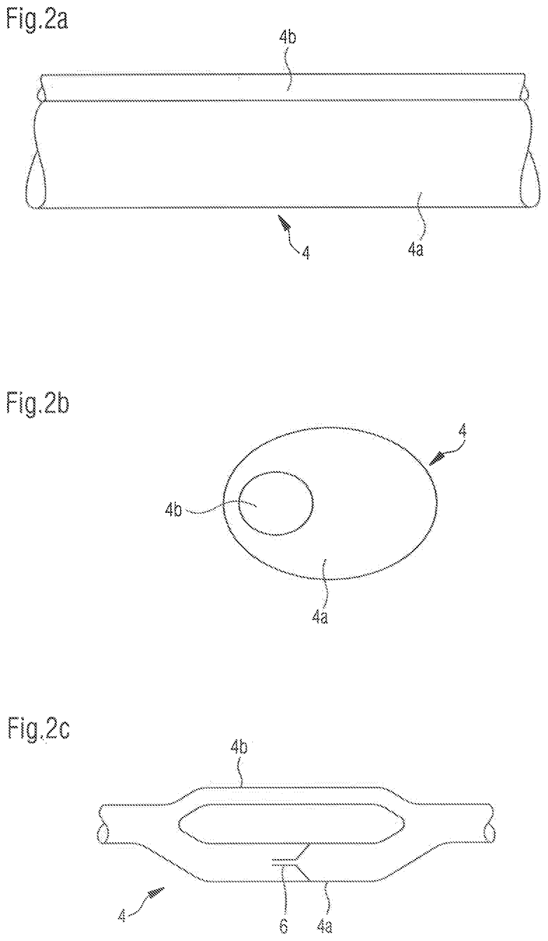

FIG. 2a shows a double-lumen hose element 4 made of film-like, easily collapsible, tubular film, having a parallel progression of the individual lumens. The supplying lumen 4b in this case has a round cross-section of 5-10 mm, preferably 6-8 mm. The draining lumen has a rounded diameter measuring 20 to 35 mm, preferably 20 to 25 mm. The tubular film body is divided in this case into two separate lumens by a longitudinally running separation, e.g., a continuous welded seam. The material used for the hose is preferably a polyurethane with a hardness of 80 A to 90 A and has a wall thickness of 150 to 300 μm, preferably 200 to 250 μm. Alternatively, the hose can be constructed of PVC, LDPE or other material with the option of forming sufficiently thin-walled, preferably easily collapsible film hoses. The function according to the invention notably requires that the hose element is capable of collapsing in a flat manner without the formation of impinging or cutting edges so that the development of pressure-related lesions can be prevented in the case that the patient lies on the hose.

FIG. 2b shows a hose element 4, wherein the lumens 4a and 4b are positioned coaxially to each other. The supplying inner hose 4b that has a smaller lumen is preferably designed in this case to be especially thin-walled in relation to the outer hose. It has a wall thickness of 30 to 150 μm, preferably of 50 to 100 μm. Because of this, the lumen 4b collapses already in the case of a small amount of force acting from the outside. The special tendency to collapse ensures that, during the phase of purging the intestinal contents, the outhowing material compresses the supplying hose and thereby frees up the entire draining lumen without any substantial space requirement. In order to prevent a space-consuming damming effect in the lumen 4b during drainage, the flow-directing valve function in the supplying leg is preferably attached at the distal end of the drainage, as shown in FIG, 1 as an example.

FIG. 2c shows a section of an embodiment of a hose element 4 for connecting a head unit SK of the catheter system 1 to a combined flushing and collecting bag 5, wherein the hose element 4 comprises an element with a flow-direction-dependent throttle function. In FIG. 2c, the hose element 4 is shown truncated at both ends, and in the disclosed section, the hose element 4 branches into two lumina 4a, 4b extending parallel to each other. One lumen 4a comprises an element 6 with a valve function, especially with a one-way valve. In the shown embodiment, this one-way valve opens if the pressure at the patient side of the element 6 (in FIG. 2c right of the element 6) is higher than the pressure at the bag side of the element 6 (in FIG. 2c left of the element 6), and it closes if the pressure difference is in the opposite direction. Therefore, a flow through the lumen 4a is only possible from the patient side or, respectively, the head unit SK of the catheter system 1, to the bag side of the element 6. In the opposite direction, the flow directing valve element 6 closes. The diameter of the lumen 4a is large enough so that even even shaped stool fractions can pass through this lumen 4a, but only in a direction from the patient or head unit SK to the bag 5.

On the other hand, a flow through the lumen 4b bypassing the lumen 4a is possible in both flow directions, but the diameter of this lumen 4b is reduced in relation to the diameter of the lumen 4a, so that the flow through lumen 4b is throttled with respect to the flow through lumen 4a.

So, alltogether, the flow in the supply direction from the bag to the patient is possible only through the lumen 4b with a throttling diameter compared to the flow in the draining direction from the patient to the bag, for which the lumen 4a with the larger diameter is open, too.

Therefore, the element 4, 6 shown in FIG. 2c has a flow-direction-dependent throttle function, which means that the throwing takes place only during a supply flow directed from the bag to the patient or head element SK, but not during a draining flow in the opposite direction.

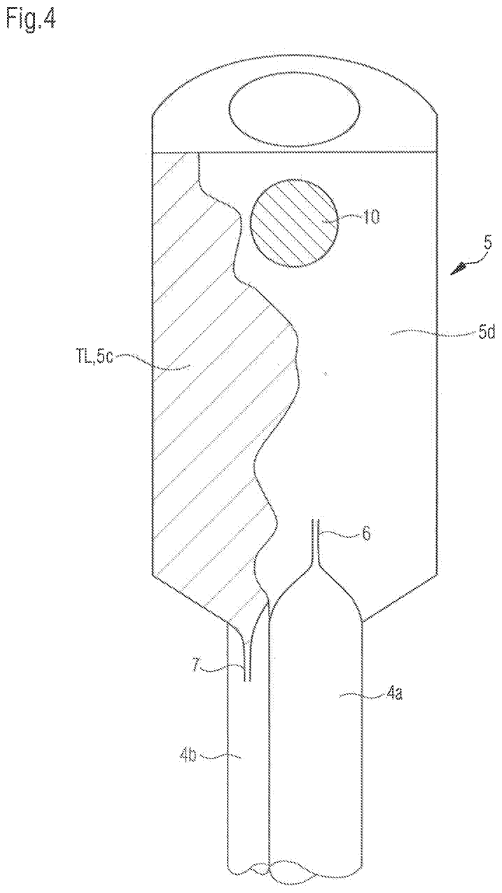

This is rather similar to the embodiment shown in FIG. 4, where the lumen 4a is blocked by the one-way valve 6 in one direction, namely from the bag 5 towards the patient, and a flow in this direction is only possible through lumen 4b with a reduced diameter compared to the lumen 4a. Therefore, FIG. 4 shows another hose element 4 comprising an element 6, 4b with a flow-direction-dependent throttle function.

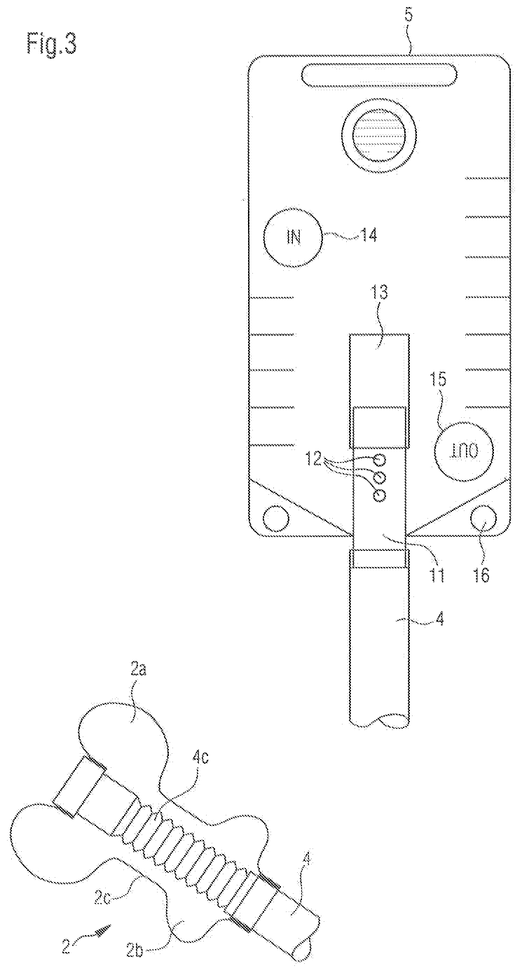

FIG. 3 shows an especially simply designed bag 5 according to the invention having a large-lumen tubular body 11 welded into the bag on the base side. The tubular body is provided with a plurality of small-lumen openings 12, through which flow can come out of the bag and be guided through the draining/supplying hose 4 towards the patient, which, however, prevent shaped fractions from flowing out of the bag. In the preferred embodiment, a thin-walled, spontaneously collapsible hose element 13 is attached on the inside end of the tubular body. Said hose element makes a valve function possible and allows only the inflow of drained material directed towards the bag, and prevents the outflow through the lumen of the tubular body. The depicted design ensures the feces-separating function according to the invention, wherein the inflow and outflow into the bag and/or the supply and drainage takes place through a single, common lumen, i.e., not separately from one another. As a result, the device requires only one single flow-directing component or function.

As another feature, the bag can be provided with an IN scale 14, which indicates the volume filled into the bag. Opposing it is an OUT scale 15, which is located in a position rotated by 180 degrees and measures the outflowing material when the bag is turned upside down following the initial repetitive flushing and draining and then can be used on a sustained basis as the collecting bag. The bag is provided with corresponding mounts 16 for this in the lower region.

The illustration furthermore shows a head unit preferably designed as a part of the invention, which is positioned in the rectum with a trans-anally sealing retention balloon 2, which comprises a rectally placed widening 2a, a preanal widening 2b and trans-anally tapered portion 2c. It also has a special shaft hose 4c bearing the balloon, which shaft hose collapses radially in an elastic manner in the case of anally applied force and spontaneously straightens when the force abates, as well as compresses like an accordion with the axial application of force and thus prevents the intestines from getting perforated.

FIG. 4 shows a completely separate, double-chamber bag or container system 5, which completely prevents a commingling of the inflowing and outflowing material. The two compartments 5c (inflow compartment) and 5d (collecting compartment) are completely spatially separated by a separating layer TL. On its lower end, the compartment 5c merges into the lumen 4b supplying the inflow. The collecting compartment 5d in this case is completely closed and cannot be evacuated. Only the inflow compartment has a filling opening. The valve functions 6 and 7 in this case are respectively ensured by valves designed to be like lips or even beaks.

In the functional transition from a double-chamber system to a single-chamber system, the separating layer TL can be configured as a mesh-like, fleece-like or sieve-like perforated element and thereby prevent the transfer of shaped stool fractions from the compartment 5d into the compartment 5c.

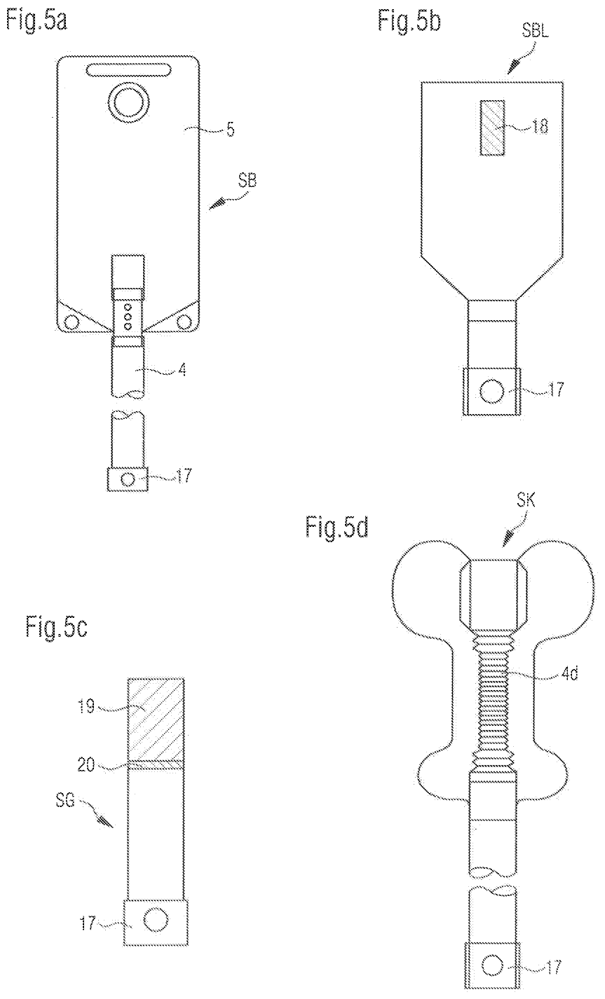

FIGS. 5a to 5e show an irrigation system for establishing or maintaining a quasi-continent state of a patient through the greatest possible removal or lavage of intestinal contents, as an overall set-based embodiment S. As components comprising the set, said embodiment, first of all, has a trans-anally sealing head unit SK, which is preferably designed in accordance with FIG. 3, and is equipped with a dumbbell-shaped, trans-anally sealing balloon and a trans-anally positioned, atraumatically foldable or compressible shaft hose, to which an elastically foldable supply and draining shaft hose 4 is attached, which merges, in turn, into a terminal connector element 17 in the relative vicinity of the head unit, preferably at a distance of approx. 20 cm from the anus.

Attached to said connector element in cyclical alternation, as another component of the set, is a bag system 5, SB for the combined high-volume irrigation and subsequent drainage, or, alternatively, a simple, lightweight collection bag SLB for receiving outgoing secretions with a degassing function according to FIG. 5b, or a gauze-filled, neck-like terminating element SG according to FIG. 5c, for primary degassing of the abdomen.

Connecting the head unit, which remains trans-anally in the patient on a sustained basis, to the respectively attached components SB, SLB and SG is preferably established by a connector element 17 whose design corresponds preferably to that of FIG. 5d. Said connector element is designed in such a way that, in the event that the patient lies on the connector, it collapses in an elastically reversible manner and/or merges into a strip-like structure that is as flat as possible and consequently prevents pressure spots.

The special structural design of the device described here as a set of product components is conceived primarily for immobile patients, who are continuously fe.cally incontinent or for sporadic, in some cases, prolonged intervals and predominantly produce feces of normal, shaped consistency. The especially high-volume, reliably trans-anally sealing lavage of the colon that can be executed with the system prevents stool from being able to form and harden in the colon, and keeps the descending colon going towards the rectum largely dear of feces, so that, in an ideal case, no feces can advance into the rectum, and thus quasi-continence is facilitated.

The method according to the invention for establishing a quasi-continence combines the following functional features of the set:

A head unit SK placed continuously on a sustained base hi the patient, which head unit facilitates, for one, a sealing of the anus against secretions or intestinal contents exiting from the rectum, and, secondly, creates a permanent large-lumen access for an intermittent, high-volume, repetitive flushing and drainage within the sense of return-flow enema;

A combined flushing and collecting bag SB, which is connected to the permanently placed head unit in approximately a two-day rhythm, and makes a repetitive return-flow enema possible, wherein the liquid supplied to the patient is preferably separated from shaped stool fractions already drained into the bag, and, following the lavage phase, the bag subsequently receives on a delayed basis the mobilized intestinal contents deposited in fractions and/or allows a closed, leak-free, multi-hour, stool drainage following the drainage; and

Optionally a lightweight, small terminating bag SLB having a secretion-receiving and degassing function, or a terminating stopper SC filled with light gauze, which, during the phases between the washouts, respectively protect from exiting secretions and intestinal contents and/or make a continuous degassing possible.

The method for achieving a quasi-continence through a high-volume washout of intestinal contents is described as follows:

After trans-anal positioning of the trans-anally sealing head unit, which drains with a large lumen, to carry out an initial savage cycle, a combined flushing and collecting bag having an optional feces-separating function and/or a correspondingly functionally designed flushing and collecting vessel is attached to the terminal connector piece of the head unit;

Following the irrigation, the bag/vessel then remains attached to the patient or to the head unit to collect subsequent stool eliminated on a delayed basis until no further elimination of mobilized stool takes place;

If stool discharge is no longer identifiable, the flushing and collecting bag is replaced with a light degassing bag or a degassing connecting piece, which remains connected to the head unit until the next irrigation cycle; and

In the case of intermittent lavage of the colon, the patient's rectum ampulla can thereby be kept in a largely feces-free state for approx. 48 hours.

FIG. 5a shows a secretion collecting bag SB according to FIG. 3, whose supply and draining hose unit 4 has a terminal connecting cone 17.

FIG. 5b shows a bag SLB having a degassing or filter element 18 integrated into the wall of the bag.

FIG. 5c shows a ventilating terminating stopper SG filled with gauze 19, which primarily vents intestinal gas and optionally also has a secretion-absorbing function, or protects the terminal gauze portion 19 from draining secretions or feces by means of a gas permeable, but liquid impermeable, layer 20.

FIG. 5d shows a head unit SK, whose design corresponds preferably to FIG. 3, the trans-anally feces-separating shaft component 4d of which preferably has a central tapering relative to the distal and proximal end of the shaft of approx. 15 to 17 mm to approx. 10 to 12 mm in diameter, and which allows an especially atraumatic, long-term trans-anal positioning of the device. In particular, the shaft hose tapered in this manner accommodating the anal canal can be reinforced by an undulated profile and/or the elastic folding and straightening properties thereof can be optimized. The amplitude and/or width of the undulation diminish in this case when transitioning from the respective terminal large lumen towards the central small lumen. The shaft component 4d should develop a profile in the anal canal that is a flat as possible, but is nonetheless easily flexible and spontaneously foldable and bendable.

FIG. 5e shows a special connector element 17 that has two optionally slightly conical cylinders 17a and 17b that are congruently pluggable into each other, made of a soft body-friendly material, which is elastically deformable and/or flatly collapsible with the moderate application of force from the outside and elastically straightening with abating force. Said material is e.g., injection-molded polyurethane with a Shore hardness of 40A-80A, preferably of 60 to 70A. The cylinders 17a and/or 17b bear annular sealing elements or sealing lips 20 applied to the contact surface, which follow the elastic deformation mechanics of the cylinders and/or corresponds thereto. The convex radius of the sealing lips preferably rests in the counterpart in correspondingly formed, receiving annular grooves, in order to produce, along with the seal, an axial affixing of the cylinders to one another and prevent the axial disconnection thereof. The sealing lips and grooves are designed such that they lift or sink from the cylinder in the smallest possible transition radii and thereby form a perpendicular angle as much as possible, which counteracts the axial disconnection in particular. An axial disconnection due to stronger tensile force applied to the connection can furthermore be prevented by cone-like, claw-like or adhesively adherent structures or elements 21 or even those provided with Velcro fasteners. In the process, the respective structures mortise or interlock both parts of the cone with each other, or connect through peel-off-able or reversibly detachable adhesive or even by hook-and-loop fixation, or for example also secure with a bayonet-like locking mechanism. In order to design the connecting cylinders even softer and/or as atraumatically as possible for the patient, the connecting component 17 can be ensheathed by a soft, preferably gel-like or gel-elastic, sock-like sleeve 22, which is e.g., firmly applied to one half of the connector or rolled up on it a sock-like manner, and from there can be unrolled onto the surface of the opposing connectors, and be applied thereto with a certain tension in a sealing and/or even adherent or reversible adhesive manner. The two halves of the connectors inserted into each other can respectively have varying wall thickness in certain circumferential regions 23, whereby it is preferred that wall thickness regions having the same wall thickness are opposite from each other. This particular manner of wall thickness distribution makes a preformed collapse of the connector possible so that its profile bends slightly and/or collapses in a flat way with pressure from the outside. In this case, the elements 21 are attached preferably in the regions having walls that are designed to be relatively thicker.

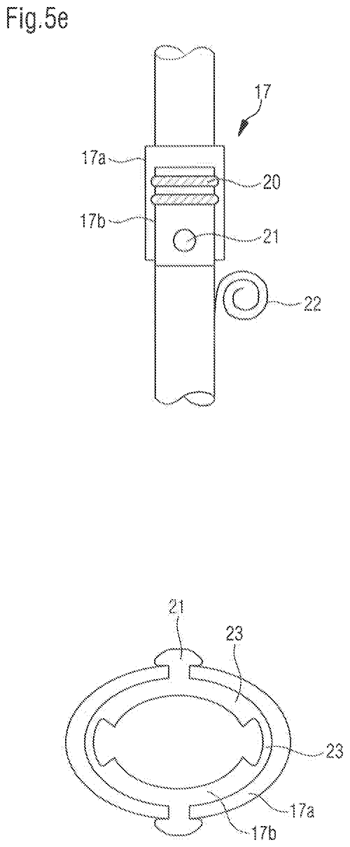

In the case of the head piece depicted in FIG. 6 of a device for carrying out a lavage-like, repetitive supply and draining of a flushing, cleaning or other therapeutically active substance, in particular in the context of a large-lumen flushing or draining cleaning of a patient's colon, an intra-rectal anchor balloon can have the form of an intra-rectal segment 101 and be fabricated together with a trans-anal intermediate segment 102 of a single, common tubular blank 108, wherein the trans-anal segment 102 of the formed balloon film 108 can merely have a one-layer wall. Even though intra-rectal and trans-anal segments 101, 102 of the balloon film 108 form one structural element in the depicted variation of the device, they have a functional separation, however, in the mounted state and in the state attached to/on a funnel element 103.

The two segments 101, 102 of the film 108 can be separated from each other and/or be separated in a sealing manner by a funnel element 103 inserted distally into the anchor balloon 101. Due to the permanent connection of the balloon film 108 in the region of the intra-rectal anchor balloon segment 101 to the funnel element 103, a fillable compartment 106 forms that can unfold like a type of collar around the lateral sections of the funnel element 103. The connection can be simplified and/or improved by specially designed preformations 107 of the funnel surface and/or the balloon film 108, which are described in more detail in the following.

Following the trans-anal segment 102 towards the proximal direction is a hose unit 105 connecting the head unit 101, 102 to a flushing and collecting bag or to a collecting and degassing unit.

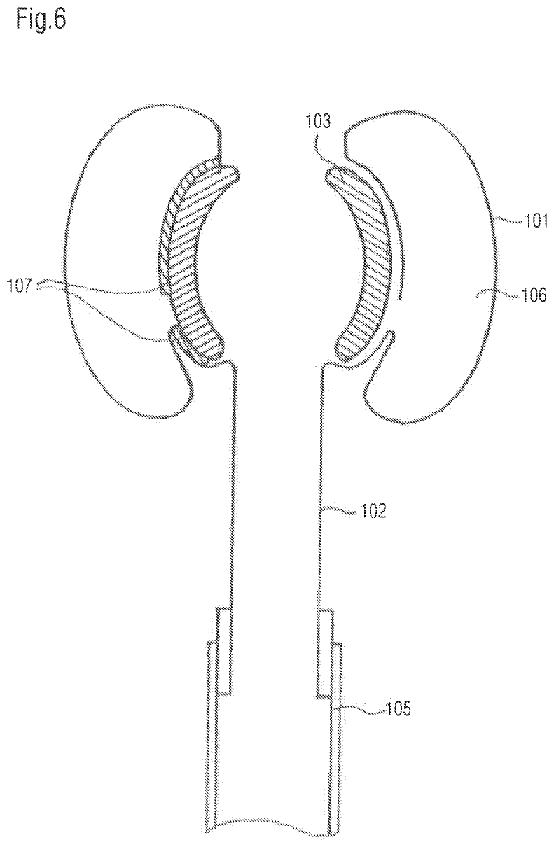

The embodiment of a head piece for a device according to the invention depicted in FIG. 7 has, instead of a proximally extended balloon end 110 emanating from anchor balloon segment 101 (as depicted e.g., in FIG. 6), a separate, trans-anally running, thin-walled tube or hose piece 125, which continuously connects the funnel 103 to the preanal connecting element or hose 104. In this case, it is an element made of an elastic material having a high restoring force, which, despite a wall designed as thin as possible, endeavors to independently straighten in the case of a radial deformation and/or to independently disentangle around the longitudinal axis thereof in the case of torsion, and thereby spontaneously return to its low-tension initial state with an open lumen

FIG. 7 shows an example of how the connection between the tube or hose piece 125 and the funnel element 103 can be designed by a preformed annular groove 124 at the proximal end of the funnel. In this case, the proximal end 126 of the intrarectal balloon sleeve segment 101 can be laid, put or guided in another way over the distal end of the tube or hose piece 125 such that same is connected in a permanently sealing manner to the funnel element 103 by inserting the distal end of the trans-anal segment 101 into the annular groove 124.

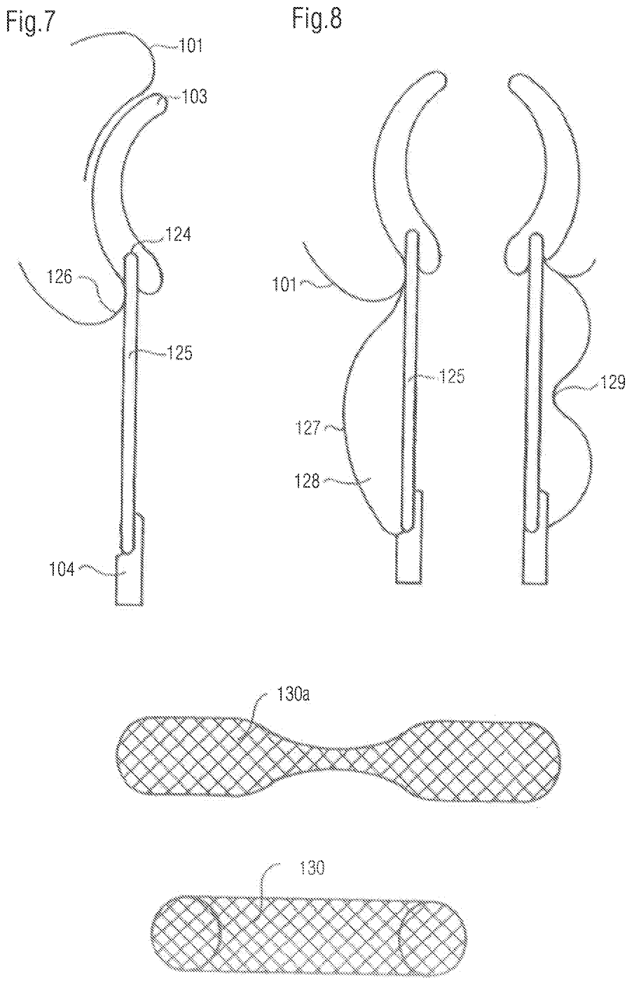

FIG. 8 shows a combination of an intra-rectal anchor balloon 101 with a trans-anal segment 102 emanating from the anchor balloon sleeve 108, as depicted in FIG. 6, with the stabilizing tube piece 125 described in FIG. 7. The trans-anal segment 102 in this case forms a separately fillable compartment 128, which forms between the inner tube or hose piece 125 and the outer proximally extended anchor balloon sleeve 127. When this compartment 128 is filled, depending on the volumetric expandability of the sleeve and/or the diameter thereof formed during manufacturing, the radial expansion of the outer sleeve 127 can improve the seal towards the anus. In addition, with forced filling, the drainage lumen of the head unit can be constricted towards the center and be substantially sealed. As an alternative to forming the sleeve 127 from the proximal offshoots of the intra-rectal anchor balloon segment 101, a separately manufactured balloon- or tubular film body can also be used for the sleeve 127. The sleeve 127 outwardly delimiting the compartment 128 can also be preformed to be shaped like a dumbbell or an hour glass. A tapering 129 that is preferably disposed approximately in the center is formed in this case such that it accommodates the structures of the anal canal. If pressure is applied to the trans-anal compartment 128, such as takes place e.g., with the instillation of irrigation liquid, the described preformation 129 ensures, firstly, a secure anchoring of the head part 101 of the device, and, secondly, contributes to sealing against the irrigation liquid being applied rectally.

The previously described techniques can be used for affixing and for the sealing termination of the components required for this embodiment in the transition area between the intra-rectal and trans-anal compartments 106, 127.

Analogously, the tubular film segment 127 preferably emanating from the intra-rectal anchor balloon segment 101 can be combined with a radially deformable tubular mesh 130 fitting snuggly under slight tension to the trans-anal contours. In a non-deformed, tension-free initial state, the tubular mesh 130 has an open lumen of approx. 2 to 3 cm. The radially directed restoring force on the anus that develops with lumen constriction is low in this case and rules out the development of pressure ulcers. If there is a tone reduction of the anal sphincter with a defecation reflex, the tubular mesh 130 follows the then opening trans-anal canal and thus facilitates the outflow of stool. The tubular mesh 130 connects the intra-rectal funnel 103 with a preanal connecting element or connecting hose 104 preferably continuously. It is preferably arranged inside the hose segment, but alternatively can also run over the outer surface thereof. In a radially constricted state 130a, the mesh 130 that is constructed preferably of rigid, flexibly deformable filaments has a considerable increase in length, which is taken into consideration by a corresponding length requirement of the surrounding trans-anal tubular film 127.

Assembling the components thereof follows the previously described techniques.

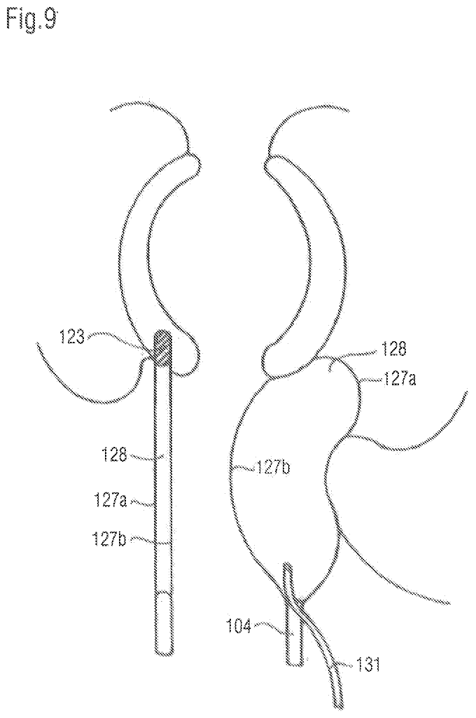

FIG. 9 represents an embodiment, the trans-anal segment of which has two concentric hose layers 127a and 127b. As previously described, they can be fabricated from the extended ends of the intra-rectal balloon segment 101. In the preferred embodiment, the inner hose layer 127b emanates from the proximal extension of the intra-rectal balloon sphere 109. The outer hose layer 127a is preferably designed as a separate thin-walled, cylindrical tubular film element or even one preformed to be congruent with the anal canal.

Alternatively, the two hose layers 127a, 127b can also be made of separately fabricated tubular films.

The space 128 that forms between the two hose layers 127a, 127b can be filled partially or completely extracorporeally with a medium via a supply 131 guided via a connecting element or connecting hose 104 or even be completely evacuated.

In an evacuated state, the two hose layers 127a, 127b fit closely next to each other and behave virtually as a one-layer wall. In a filled state, the two layers 127a, 127b separate from each other, and the drainage lumen is opened to a maximum.

In a bulgingly filled state, the inner layer 127b expands towards the center of the drainage lumen and seals it so that it is impervious to liquid. The outer layer 127a, on the other hand, expands towards the anal wall and fits snugly against it, likewise in a sealing manner following the respective anatomy.

In the case of partial filling or filling with a few milliliters of filling medium, the two layers 127a, 127b separate. The two thin-walled film layers 127a, 127b slide freely displaceably to each other, virtually supported by the filling medium, Due to the free play and the sliding of the film layers 127a, 127b, lesions in the especially sensitive region of the anal canal, such as those known from statically abutting, less dynamic films, can be better prevented.

Connecting the components thereof to the proximal end of the funnel can take place on the basis and/or by means of an annular support 123, which is inserted into an annular preformation 124 on the proximal rim of the funnel. In this case, the inner layer 127b can be guided via the ring 123, as in FIG. 6c, and be connected to the funnel 103. In a preceding assembly step, the outer layer 127a can be affixed on its distal end to the ring 123.

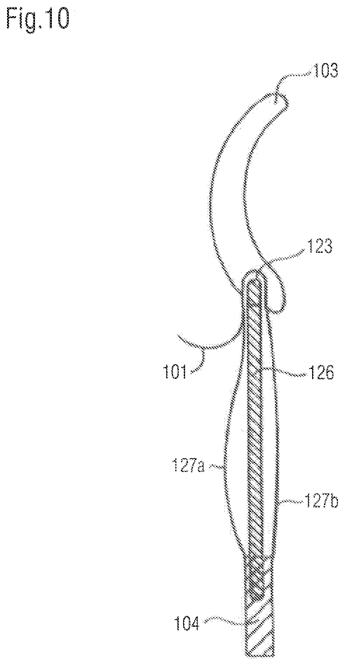

FIG. 10 combines the previous described embodiment with the elastically deformable, self-straightening and aligning tubular mesh 130 described in FIG. 8. The previously described hose or tube element 125 can be used as an alternative to a tubular mesh 130.

The previously described elements 125, 130 are preferably installed between the two film layers 127a and 127b, connecting the funnel 103 to a connecting element or connecting hose 104.

In the case of all embodiments according to FIG. 6 to FIG. 10, the funnel element 103 can also be situated inside the intra-rectal compartment 106, i.e., between the outer film layer 109 and the inner layer and/or compartment 111 of the intra-rectal anchor balloon segment 101.

All these head parts 101, 102 according to one of the FIGS. 6 to 10 can be coupled via a tubular film element 4 to a flushing and collecting bag 5 according to one of the FIGS. 1 to 5. In addition, an element with a flow-directing function can be situated in the region of each of these head parts 101, 102, preferably in a hose element provided for the supply of flushing or irrigation liquid to the patient.

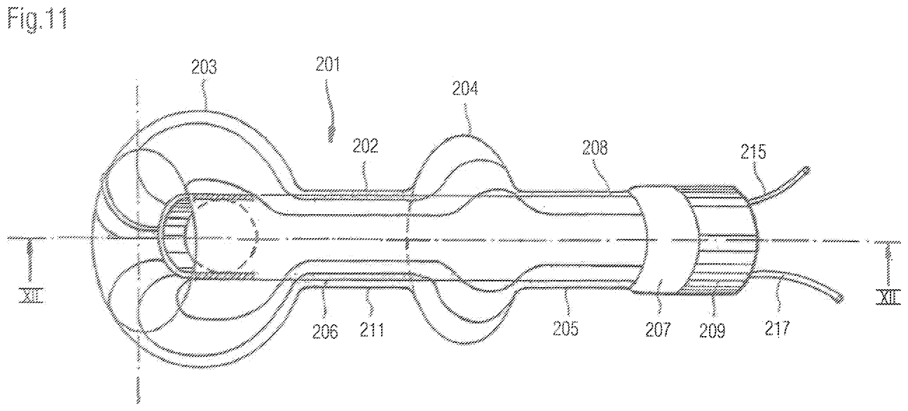

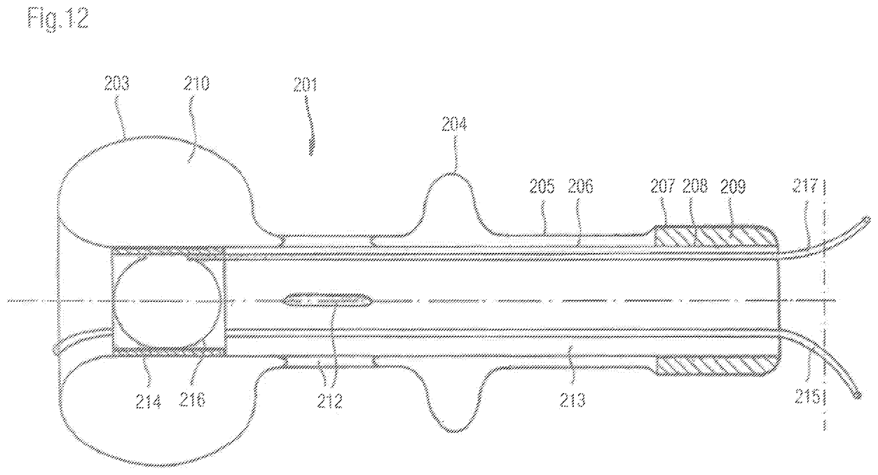

FIGS. 11 and 12 depict another head unit for a device 201 according to the invention for carrying out a lavage-like, repetitive supply and draining of a flushing, cleaning or other therapeutically active substance, in particular in the context of a large-lumen flushing or drainage of a patient's colon, which can likewise be coupled via a tubular film element 4 to a flushing and collecting bag 5 according to one of the FIGS. 1 to 5. In this case as well, an element having a flow-directing function can be situated in the region of each head part 201, preferably in a hose element provided for the supply of flushing or irrigation liquid to the patient.

The device 201 is used for flushing and/or sealing a natural or artificial outlet from the bowel. It comprises a preformed hose 202 that is folded back on itself made of a thin-walled material, for example polyurethane with a Shore hardness degree of A 90 and a wall thickness of less than 25 μm.

The hose 202, whose original diameter is approximately between 15 mm and 30 mm, obtains two radial widenings 203, 204 as a result of the preformation. The one, preferably larger, widening 203 is located approximately in the center of the hose, which forms the patient-proximal end of the device 201 after fold-over. The other preformation 204 is located approximately in the center of the outward hose section 205 after the fold-over, while the inward hose section 206 does not have this kind of widening, but has a constant cross-section.

One or both of the free ends 207, 208 of the two hose sections 205, 206 can be connected to a connector piece 209, which is sleeve-shaped for example. The connector piece 209 can have, in particular in the region of its patient-distal end, an internal and/or external thread for connecting various medical apparatuses, for example a flushing and collecting bag 5, an extracorporeal degassing unit or the like.

It is preferred that the outer layer 205 of the balloon 202 be adhesively bonded to the exterior of the connector piece 209, and the inner layer 206 of the balloon 202 can be adhered to the inside thereof. As a result, the cavity 210 between the inner and outer layers 205, 206 of the balloon 202 is sealed in an airtight manner; located only in the region of the connector piece 209 is a connection to the outside (not shown in the drawing) to which a source with a medium that can be pressurized can be attached for deploying the balloon 202. Instead of a sleeve-shaped connector piece 209, the two balloon layers 205, 206 can also be fastened directly to each other, for example adhered together.

Due to the relatively great material hardness of the balloon 202, said balloon is only marginally elastic and, in an inflated state, assumes its shape predetermined by the preformation that is recognizable in FIG. 11. Said shape has an approximately cylindrical basic shape having an approximately spherical widening 203 at the patient-proximal end opposite from the connector piece 209 or another closure there and having an annular or discoid widening 204 approximately in the center between the two ends 203, 209.

The spherical widening 203 is placed in a deflated state in the patient's rectum (indorectal section), while the subsequent, cylindrical section 211 up to the annular or discoid widening 204 leads through the anal canal to the outside (transrectal region), [and] the patient-distal widening 204 is located in the anal fold.

In the transrectal region 211 between the two widenings 203, 204, the two layers of the balloon 202, namely the outer layer 205 and the inner layer 206, are connected to one another, preferably by welds 212 or bonds. In doing so, these can be punctiform, linear or laminar welds, The depicted embodiment foresees four welding lines 212 running in the axial direction, each of which is offset from one another by approximately the same circumferential angle. Thanks to said welded connections 212, the inner lumen 213 within the inner layer 206 of the balloon 202 can open more easily when the intestines are supposed to be evacuated.

In the region of the patient-proximal widening 203, the inner lumen 213 is kept open by a reinforcing sleeve 214, the length of which is preferably the less than or equal to the axial extension of the radial, indorectal widening 203 of the balloon 202. The material hardness of the reinforcing sleeve 214 is preferably less than or equal to the material hardness of the balloon; the sleeve 214 obtains its rigidity from its increased wall thickness. The same material as for the hose or balloon 202 can preferably be used for the sleeve 214. This facilitates the affixing of the sleeve 214 inside the inner lumen 213, in particular by adhesion with the inner layer 206 of the balloon 202, wherein, in such a case, a means solubilizing the material in question can be used as the adhesive or for welding.

A hose 215 extends preferably inside the inner lumen 213 from the patient-proximal end 203 to other side of the connector piece 209. An irrigation medium can be introduced into the patient's intestines through said hose 215, which can be affixed to the balloon 202, preferably on the inner layer 206 thereof, for example by adhesion. The opening of the hose 215 is located at the forward-most region of the indorectal end 203 of the device 201 so that, in such a case, no bacteria can be washed into the intestines from the transrectal region 211 or from the region on the other side of the transrectal widening 204. The hose 215 can be fed internally through the reinforcing sleeve 214 or between the same and the inner layer 206.

An occlusion balloon 216 is also provided in order to be able to optimally block the central lumen 213 during an inflow. Said balloon preferably sits inside the reinforcing sleeve 214 and, in the case of the embodiment according to FIGS. 11 and 12, has a spherical performation with a diameter that is somewhat larger than the diameter of the reinforcing sleeve 214. As a result, the inflated occlusion balloon 216 seals the central lumen 213 completely and, when under pressure as well as under less deformation all around, rests against the inside reinforcing sleeve 214 thereby sealing same.

Discharging at the occlusion balloon 216 is another hose 217, with which a preferably liquid pressure medium can be guided into the occlusion balloon 216 in order to seal the lumen 213 in the region of the reinforcing sleeve 214. The hose 217 is guided through the connector piece 209 on the inside of the balloon inner layer 206, and is therefore accessible from the outside. Approximately in the region of the discharging of the hose 217 into occlusion balloon 216, the latter is affixed to the reinforcing sleeve 214, for example adhered approximately in a punctiform manner.

A short hose piece can be formed on the respective head unit 2, 101, 102, 201, 201′, which hose piece then can merge into or discharge into a hose 4, whereby then there is proximally a connector 17 for optionally connecting a flushing and collecting bag 5 or a collecting and degassing unit or a further peripheral element.

FIGS. 6 to 12 are supposed to document that the system according to the invention and especially the device according to the invention can be used with different head units. While in FIG. 1 a simple, preferably intrarectally placeable head unit can be used, which, similar to FIG. 6, consists of only a single balloon compartment, which is accommodated in situ completely in the rectum, while the trans-anal section consists only of a single hose layer of this balloon, in the case of the embodiment according to FIG. 7, a separate, preferably reinforcing element is used, In the case of FIG. 8, a hose layer of the balloon is used in the trans-anal section, on the one hand, with a reinforcing element that is separate therefrom. The embodiment according to FIG. 9 has two concentric two hose layers in the trans-anal region, which delimit a compartment, which is completely separate from the intra-rectal balloon compartment and, therefore, can be filled independently of the other compartment, for example in order to achieve a sealing. In the case of the embodiment according to FIG. 10, a reinforcing element is also included within the trans-anal compartment, which is completely separate from the intrarectal compartment. On the other hand, in the case of the head unit 201, the intermediate space remaining the between the concentric layers of the hose section folded back to form a balloon is not divided into two compartments, rather the intra-rectal cavity communicates with the trans-anal cavity. On the other hand, the two hose layers can be welded to each other in a punctiform, linear or laminar manner in order to keep a draining lumen open for example.

Although all head units according to FIGS. 6 to 12 can be used for a non-leaking, contamination-free mobilising and removal of intestinal contents of higher poritions of the colon, as well as for an optional subsequent non-leaking, temporarily determinable instillation of high volumes of lactulose solution, especially in the case of a therapy of a hepatic encephalopathy, embodiments according to FIGS. 7 to 12 are preferred, because a mushroom-shaped or dumbbell-shaped balloon can accomplish an optimum sealing in the anus in order to prevent a leakage during the therapy. These embodiments are able to spontaneously adapt to the motility of the anal closure apparatus or body movements of the patient in order to achieve a very tight sealing. Although the balloon structure according to FIGS. 8 and 9 comprise two balloon compartments separated from each other, this is no necessary feature, and a balloon structure according to FIGS. 11 and 12 with only one common compartment would be sufficient for a tight sealing of the anus.

List of Reference Numbers

| | |
|---|---|
| 1 | flushing catheter system |
| 2 | retention balloon |
| 2a | rectal widening |
| 2b | preanale widening |
| 2c | trans-anal tapering |
| 3 | central opening |
| 4 | tubular film element |
| 4a | draining leg |
| 4b | supplying leg |
| 4c | shaft hose |
| 4d | shaft component |
| 5 | flushing and collecting bag |
| 5c | compartment |
| 5d | compartment |
| 6 | valve-like unit |
| 7 | functional element |
| 8 | inlet element |
| 9 | mesh-like separating film |
| 10 | sealable opening |
| 11 | tubular body |
| 12 | openings |
| 13 | hose element |
| 14 | IN scale |
| 15 | OUT scale |
| 16 | mount |
| 17 | connector element |
| 17a | cylinder |
| 17b | cylinder |
| 18 | filter element |
| 19 | gauze |
| 20 | sealing lip |
| 21 | element |
| 22 | sleeve |
| 23 | circumferential regions |
| 101 | intrarectal balloon segment |
| 102 | trans-anal balloon segment |
| 103 | funnel element |
| 104 | connecting hose |
| 105 | evacuation hose |
| 106 | compartment |
| 107 | preformation |
| 123 | ring-like Element |
| 124 | annular groove-like preform |
| 125 | tube or hose piece |
| 126 | proximal end |
| 127 | outer sleeve, tubular film |
| 128 | compartment |
| 129 | tapering |
| 130 | tubular mesh |
| 131 | supply |

201 device

202 preformed hose

203 radial widening

204 radial widening

205 hose section

206 hose section

207 free end

208 free end

209 connector element

210 cavity

211 cylindrical section

212 welded connection

213 inner lumen

214 reinforcing sleeve

215 hose

216 occlusion balloon

217 hose

218 hose

219 inner sleeve

220 canal

S set-based embodiment

SB flushing and collecting bag

SG terminating element

SK head unit

SLB collection bag

1. A device for carrying out a lavage-like, repetitive supply and draining of a flushing, cleaning or other therapeutically active substance in the context of a return-flow enema, the device comprising:
a catheter unit (SK) for receiving and draining feces, which can be permanently placed atraumatically and anorectally;
a flushing and collecting bag unit (5, SB) with a combined flushing and collecting function; and
a hose unit (4) connecting the catheter unit (SK) to the flushing and collecting bag unit (5, SB), comprising a single-lumen or multi-lumen, film-like, flatly collapsible, hose, and enabling a bidirectional flow communication between the catheter unit (SK) and the flushing and collecting bag unit (5; SB);
characterized by at least one throughflow element with a flow-direction-dependent throttle function, providing a large lumen for draining of the patient's intestinal contents into the flushing and collecting bag unit (5; SB), so that shaped stool fractions can be drained, and which facilitates a supply of liquid from the flushing and collecting bag unit (5; SB) towards the patient, but wherein the supply of liquid towards the patient is throttled, so as to be reduced in flow amount or flow speed in relation to the draining of the patient's intestinal contents and thereby reduces the supply of liquid to the patient to a non-irritating amount that is compatible with the patient avoiding painful sensations and/or vegetative responses by an immediate dilation of the patient's colon and/or a triggering of a premature or only partially coordinated defecation reflex.

2. The device according to claim 1, characterized in that both the inward flow from the flushing and collecting bag unit (5, SB) towards the patient, as well as the outward flow from the patient to the flushing and collecting bag unit (5, SB) are flow-directed by corresponding elements selected from the group consisting of valves, one-way valves, and valve-like functional or control elements.

3. The device according to claim 1, characterized in that the outward flow from the patient to the flushing and collecting bag unit (5, SB) is flow-directed by a corresponding element selected from the group consisting of a valve, a one-way valve, and a valve-like functional or control element.

4. The device according to claim 1, characterized in that the supply of liquid takes place either via the large lumen as for the draining of the patient's intestinal contents, or by a separate lumen having a smaller cross-section relative to the large lumen for the draining of the patient's intestinal contents.

5. The device according to claim 1, characterized by an element with a feces-separating function, which allows shaped fractions in the bag unit (5, SB) to be separated out of the intestinal contents already drained into the bag unit (5, SB) and to keep them captive there and/or merely allows return flow of liquid fractions of the repetitively supplied and drained substance from the bag unit (5, SB) to the patient.

6. The device according to claim 5, characterized in that the separating element has a mesh-like, a web-like or a sieve-like perforated structure, which allows liquid or feces suspended in liquid to pass through, but prevents formed feces fractions from flowing back to the patient.

7. The device according to claim 5, characterized in that the bag unit (5, SB) has a separating layer (TL), which either completely separates two compartments (5c, 5d) from each other, or is configured as a feces-separating or feces-filtering, mesh-like, fleece-like or sieve-like perforated structure, which separates liquid feces fractions from solid ones.

8. The device according to claim 5, characterized in that an element (12) with a flow-reducing function and an element (13) with a flow-directing function are integrated or disposed inside the bag unit (5, SB), in the terminal extension of the connecting hose unit (4) having a single-lumen, combined supply and draining structure.

9. The device according to claim 1, characterized in that the catheter unit (SK) passing through and sealing an anal canal of the patient over the entire length thereof comprises a single-walled or a double-walled balloon component (2), preferably made of polyurethane formed from blow molding to tho anatomically required dimensions.

10. The device according to claim 9, characterized in that the single-walled or a double-walled balloon component (2) is configured to be dumbbell-shaped on an exterior thereof, such that a waist-like tapering is configured to pass through the anal canal of the patient, while a distal widening is configured to be disposed intrarectally and a proximal widening is configured to be disposed extracorporeally.

11. The device according to claim 9, characterized in that a hose component (4c, 4d), which is feces-draining, can be placed trans-anally and bears the balloon component, is disposed inside a dumbbell-shaped balloon component (2), wherein the hose component (4c, 4d) is reinforced and therefore facilitates comparable elastic straightening properties with lower wall thicknesses in relation to a hose which is formed in a non-reinforced manner of the same diameter and material.

12. The device according to claim 1, characterized in that the connecting hose unit (4) and/or a connector (17) disposed on a proximal end thereof is flatly collapsible from outside atraumatically with a slight exertion of force in order to thereby prevent the development of pressure spots in an event that the patient lies on the connecting hose unit (4) and/or the connector (17).

13. The device according to claim 1, characterized in that, during a return-flow enema, enables the flushing and collecting bag unit (5, SB) (i) to be lifted above a patient's level for supplying a liquid towards the patient, and (ii) to be lowered below the patient's level for draining of the patient's intestinal contents.

14. The device according to claim 1, characterized in that the at least one element with a flow-direction-dependent throttle function facilitates a supply of liquid towards the patient, but wherein the supply of liquid towards the patient takes place through a component with a throttling dimensioning, in order to reduce the flow amount or flow speed of the liquid supplied towards the patient in relation to the flow amount or flow speed of a medium drained from the patient.

15. The device according to claim 12, characterized by an extracorporeal collecting and/or degassing unit that can be optionally connected to the connector element (17).

16. The device according to claim 1, characterized in that the catheter unit (SK) comprises a balloon component (2) which is has a distal balloon segment which is to be placed intrarectally and a tapered balloon segment proximally adjacent thereto, which is to be placed trans-anally in order to seal the anus.

17. The device according to claim 16, characterized in that that the catheter unit (SK) furhter comprises, proximally adjacent to the tapered balloon segment, a proximal balloon segment of a larger radius than the tapered balloon segment, thereby providing a waist-like tapering between the distal balloon segment and the proximal balloon segment, which is configured to pass through the anal canal of the patient, while the widened distal balloon segment is configured to be disposed intrarectally and the widened proximal balloon segment is configured to be disposed extracorporeally.

18. A method for carrying out a lavage-like, repetitive supply and draining of a flushing, cleaning or other therapeutically active substance in the context of a return-flow enema, with the following steps:
a) providing a device comprising:
a catheter unit (SK) for receiving and draining feces, which can be permanently placed atraumatically and anorectally;
a flushing and collecting bag unit (5, SB) with a combined flushing and collecting function;
a hose unit (4) connectable between the catheter unit (SK) and the flushing and collecting bag unit (5, SB), comprising a single-lumen or multi-lumen, film-like, flatly collapsible, hose and enabling a bidirectional flow communication between the catheter unit (SK) and the flushing and collecting bag unit (5; SB); and
at least one throughflow element with a flow-direction-dependent throttle function, wherein the throughflow element is arranged in one of the catheter unit (SK), the flushing and collecting bag unit (5, SB) or the hose unit (4) or between two of them, and comprises a large lumen for draining of the patient's intestinal contents into the flushing and collecting bag unit (5; SB), so that shaped stool fractions can be drained, and facilitates a supply of liquid from the flushing and collecting bag unit (5; SB) towards the patient, but wherein the supply of liquid towards the patient is throttled, so as to be reduced in flow amount or flow speed in relation to the draining of the patient's intestinal contents and thereby reduces the supply of liquid to the patient to a non-irritating amount that is compatible with the patient avoiding painful sensations and/or vegetative responses by an immediate dilation of the patient's colon and/or a triggering of a premature or only partially coordinated defecation reflex;
b) insertion of the the catheter unit (SK) into the natural or artificial opening into the colon of the patient;
c) connecting the flushing and collecting bag unit (5, SB) to the catheter unit (SK) via the hose unit (4) and the throughflow element;
d) supplying liquid from the flushing and collecting bag unit (5, SB) towards the patient by raising the flushing and collecting bag unit (5, SB) to a level above the intestine of the patient, wherein the supply of liquid towards the patient is throttled by the throughflow element, so as to be reduced in flow amount or flow speed in relation to the draining of the patient's intestinal contents and thereby reduces the supply of liquid to the patient to a non-irritating amount that is compatible with the patient;
e) draining liquid and the patient's intestinal contents from the patient to the flushing and collecting bag unit (5, SB) by lowering the flushing and collecting bag unit (5, SB) to a level beneath the intestine of the patient, wherein the throughflow element provides a large lumen for draining shaped stool fractions together with the other intestinal contents of the patient;
f) repeating the above steps d) and e) until the descending colon of the patient is evacuated of feces.

19. The method according to claim 18, wherein step f) is followed by another step:
g) lowering the flushing and collecting bag unit (5, SB) to a level beneath the intestine of the patient with the connector element (17) of the flushing and collecting bag unit (5, SB) directed upwards in order to drain residual liquid from the patient to the flushing and collecting bag unit (5, SB).

20. The method according to claim 18, wherein step f) or a following step g) is followed by another step:
h) leaving the catheter unit (SK) inserted in the anus to ensure a continuous sealing of the anus over the interval up to the next irrigation, wherein the catheter unit (SK) is terminated with either a lightweight, film-like secretion collecting bag or by a degassing element filled with gauze-like or absorbent cotton-like material.

21. The method according to claim 18, wherein after step f) or after a following step g) or h), an incontinent patient temporarily achieves a state of a so-called quasi-continence.

22. The method according to claim 18, wherein step f) or a following step g) is followed by another step:
i) preparation of a lactulose solution, filling the lactulose solution into a flushing and collecting bag unit (5, SB), connecting this flushing and collecting bag unit (5, SB) to the catheter unit (SK) of the patient via the hose unit (4), and subsequently flushing infusion of the lactulose solution into the colon via the catheter unit (SK).

23. The method according to claim 22, wherein step i) is followed by another step:
j) closing of the hose unit (4) for a temporarily determined exposition of the lactulose solution in the colon.

24. The method according to claim 23, wherein step j) is followed by another step:
k) lowering the flushing and collecting bag unit (5, SB) to a level beneath the intestine of the patient with the connector element (17) of the flushing and collecting bag unit (5, SB) directed upwards in order to drain residual lactulose solution and, if applicable, the patient's intestine contents, from the patient without contamination to the flushing and collecting bag unit (5, SB).

25. The method according to claim 18, wherein the hose unit (4) is used to connect the secretion collecting bag to the catheter unit (SK), too.

26. The method according to claim 18, wherein the connection of the catheter unit (SK) to the flushing and collecting bag unit (5, SB) or to the secretion collecting bag takes place close to the patient's body at a distance from the anus of 30 cm to 80 cm.

27. The method according to claim 18, wherein both the inward flow from the flushing and collecting bag unit (5, SB) towards the patient, as well as the outward flow from the patient to the flushing and collecting bag unit (5, SB) are flow-directed by corresponding elements selected from the group consisting of valves, one-way valves, and valve-like functional or control elements.

28. The method according to claim 18, characterized in that the outward flow from the patient to the flushing and collecting bag unit (5, SB) is flow-directed by a corresponding element selected from the group consisting of a valve, a one-way valve, and a valve-like functional or control element.

29. The method according to claim 18, characterized in that the supply of liquid takes place either via the large lumen for the draining of the patient's intestinal contents, or by a separate lumen having a smaller cross-section relative to the large lumen for the draining of the patient's intestinal contents.

30. The method according to claim 18, characterized by an element with a feces-separating function, which allows shaped fractions in the bag unit (5, SB) to be separated out of the intestinal contents already drained into the bag unit (5, SB) and to keep them captive there and/or merely allows return flow of liquid fractions of the repetitively supplied and drained substance from the bag unit (5, SB) towards the patient.

31. The method according to claim 30, characterized in that the separating element has a mesh-like, a web-like or a sieve-like perforated structure, which allows liquid or feces suspended in liquid to pass through, but prevents formed feces fractions from flowing back to the patient.

32. The method according to claim 30, characterized in that the bag unit (5, SB) has a separating layer (TL), which either completely separates two compartments (5c, 5d) from each other, or is configured as a feces-separating or feces-filtering, mesh-like, fleece-like or sieve-like perforated structure, which separates liquid feces fractions from solid ones.

33. The method according to claim 30, characterized in that an element (12) with a flow-reducing function and an element (13) with a flow-directing function are integrated or disposed inside the bag unit (5, SB), in the terminal extension of the connecting hose unit (4) having a single-lumen, combined supply and draining structure.

34. The method according to claim 18, characterized in that the catheter unit (SK) passing through and sealing an anal canal of the patient over the entire length thereof comprises a single-walled or a double-walled balloon component (2), preferably made of polyurethane formed from blow molding to tho anatomically required dimensions.

35. The method according to claim 34, characterized in that the single-walled or a double-walled balloon component (2) is configured to be dumbbell-shaped on an exterior thereof, such that a waist-like tapering is configured to pass through the anal canal of the patient, while a distal widening is configured to be disposed intrarectally and a proximal widening is configured to be disposed extracorporeally.

36. The method according to claim 34, characterized in that a hose component (4c, 4d), which is feces-draining, can be placed trans-anally and bears the balloon component, is disposed inside a dumbbell-shaped balloon component (2), wherein the hose component (4c, 4d) is reinforced and therefore facilitates comparable elastic straightening properties with lower wall thicknesses in relation to a hose which is formed in a non-reinforced manner of the same diameter and material.

37. The method according to claim 18, characterized in that the connecting hose unit (4) and/or a connector (17) disposed on a proximal end thereof is flatly collapsible from outside atraumatically with a slight exertion of force in order to thereby prevent development of pressure spots in an event that the patient lies on the connecting hose unit (4) and/or the connector (17).

38. The method according to claim 37, characterized by an extracorporeal collecting and/or degassing unit that can be optionally connected to the connector element (17).

39. The method according to claim 18, characterized in that the catheter unit (SK) comprises a balloon component (2) which is has a distal balloon segment which is to be placed intrarectally and a tapered balloon segment proximally adjacent thereto, which is to be placed trans-anally in order to seal the anus.

40. The device according to claim 39, characterized in that that the catheter unit (SK) furhter comprises, proximally adjacent to the tapered balloon segment, a proximal balloon segment of a larger radius than the tapered balloon segment, thereby providing a waist-like tapering between the distal balloon segment and the proximal balloon segment, which is configured to pass through the anal canal of the patient, while the widened distal balloon segment is configured to be disposed intrarectally and the widened proximal balloon segment is configured to be disposed extracorporeally.
